(19) United States
(12) Patent Application Publication
(10) Pub. No.: US 2021/0000629 A1
(43) Pub. Date: Jan. 7, 2021

(54) ORTHOSIS

(71) Applicant: YU-CHIEN WANG, Taichung City (TW)

(72) Inventor: YU-CHIEN WANG, Taichung City (TW)

(21) Appl. No.: 16/503,024

(22) Filed: Jul. 3, 2019

(51) Int. Cl. A61F 5/01; A61F 5/14

(52) U.S. Cl. CPC A61F 5/0111; A61F 5/019; A61F 2005/0181; A61F 2005/0132; A61F 5/14

(57) ABSTRACT

An orthosis is provided, including: a first supporting member, a second supporting member, a pivot mechanism and an attachment mechanism. The first supporting member is configured to be attached to an inner side of a foot and correspond to a big toe. The second supporting member is configured to be attached to the inner side of the foot and correspond to a metatarsophalangeal (MTP) joint of the big toe. The first supporting member and the second supporting member are rotatably connected with the pivot mechanism, and the pivot mechanism has a pivot axis configured to correspond to a distal interphalangeal (DIP) joint of the big toe. The attachment mechanism is configured to attach the first supporting member and the second supporting member to the foot.

### BACKGROUND OF THE INVENTION

### Field of the Invention

The present invention relates to an orthosis.

### Description of the Prior Art

Hallux valgus is a common foot deformity of the metatarsophalangeal (MTP) joint of a big toe. It is usually results from intrinsic or extrinsic reasons, such as systematic disease, severe flatfoot or footwear (tight pointed shoes). Common problems in people with hallux valgus include foot pain caused by swelling and inflammation of the MTP joint of the big toe, which may become severe especially when wearing inadequate shoes. Therefore, an orthosis for supporting the big toe is developed to correct the deformation of the MTP joint of the big toe and reduce foot pain.

However, a conventional orthosis includes two supporting boards which are rotatably connected with a shaft configured to correspond to the MTP joint, and the conventional orthosis is fixed on the big toe and the forefoot. Therefore, the distal interphalangeal (DIP) joint of the big toe is unable to rotate when wearing the conventional orthosis, which obstructs the user's movement and reduce wearing comfortability.

The present invention is, therefore, arisen to obviate or at least mitigate the above-mentioned disadvantages.

### SUMMARY OF THE INVENTION

The main object of the present invention is to provide an orthosis which allows rotations of joints of a big toe and is comfortable for wearing.

To achieve the above and other objects, the present invention provides an orthosis, including: a first supporting member, a second supporting member, a pivot mechanism and an attachment mechanism. The first supporting member is configured to be attached to an inner side of a foot and correspond to a big toe. The second supporting member is configured to be attached to the inner side of the foot and correspond to a metatarsophalangeal (MTP) joint of the big toe. The first supporting member and the second supporting member are rotatably connected with the pivot mechanism, and the pivot mechanism has a pivot axis configured to correspond to a distal interphalangeal (DIP) joint of the big toe. The attachment mechanism is configured to attach the first supporting member and the second supporting member to the foot.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of a preferable embodiment of the present invention;

FIG. 2 is a breakdown drawing of a preferable embodiment of the present invention;

FIG. 3 is a partial cross-sectional view of a preferable embodiment of the present invention;

FIG. 4 is a schematic diagram of a preferable embodiment of the present invention in use;

FIG. 5 is a partial cross-sectional view of a preferable embodiment of the present invention in use;

FIG. 6 is a stereogram of another preferable embodiment of the present invention.

### DETAILED DESCRIPTION OF THE PREFERRED EMBODIMENTS

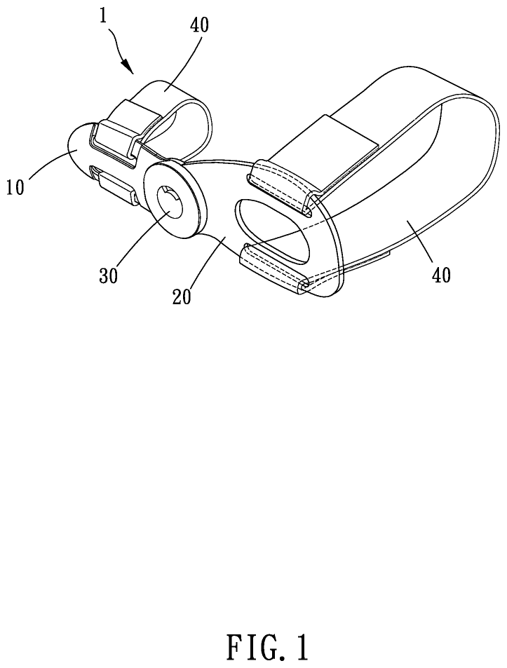

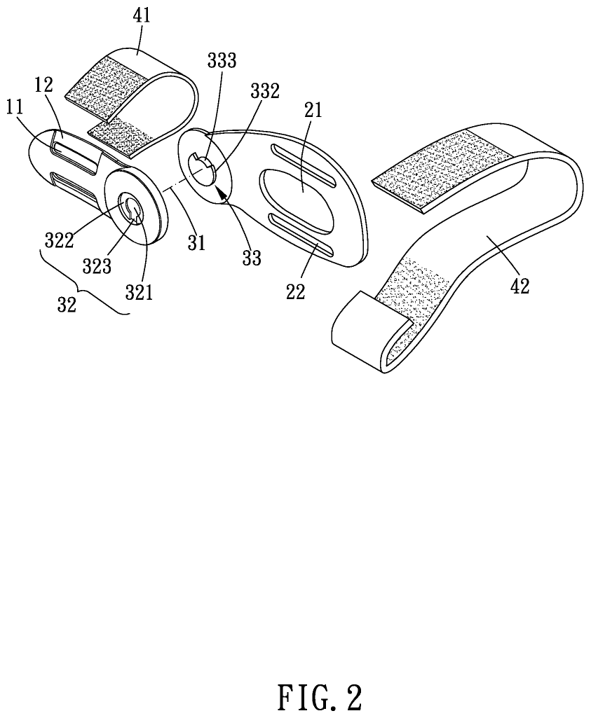

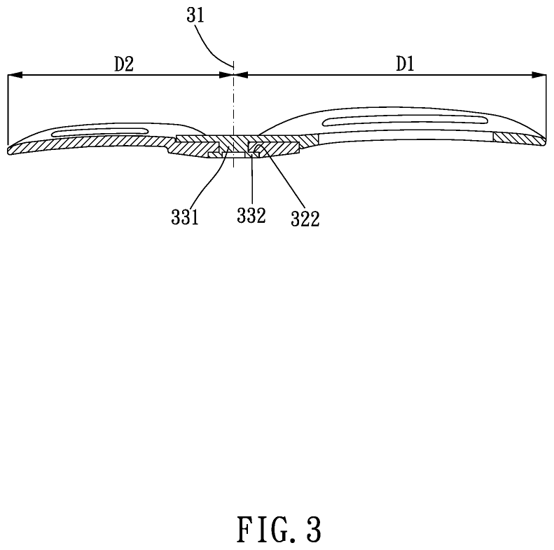

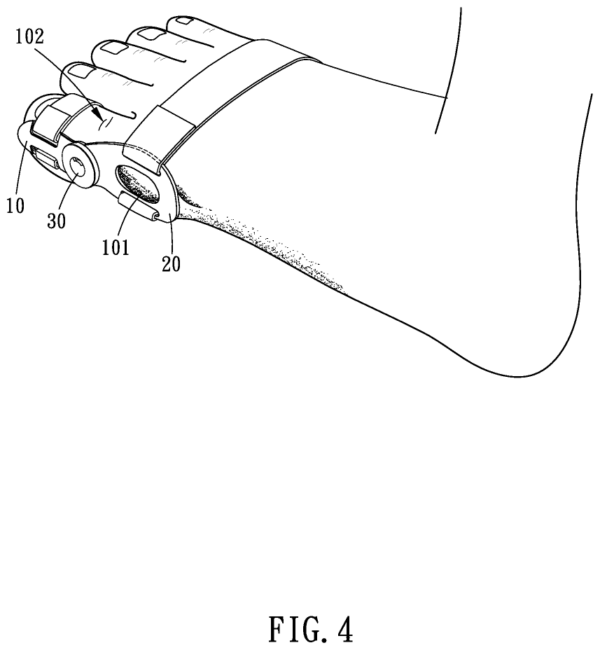

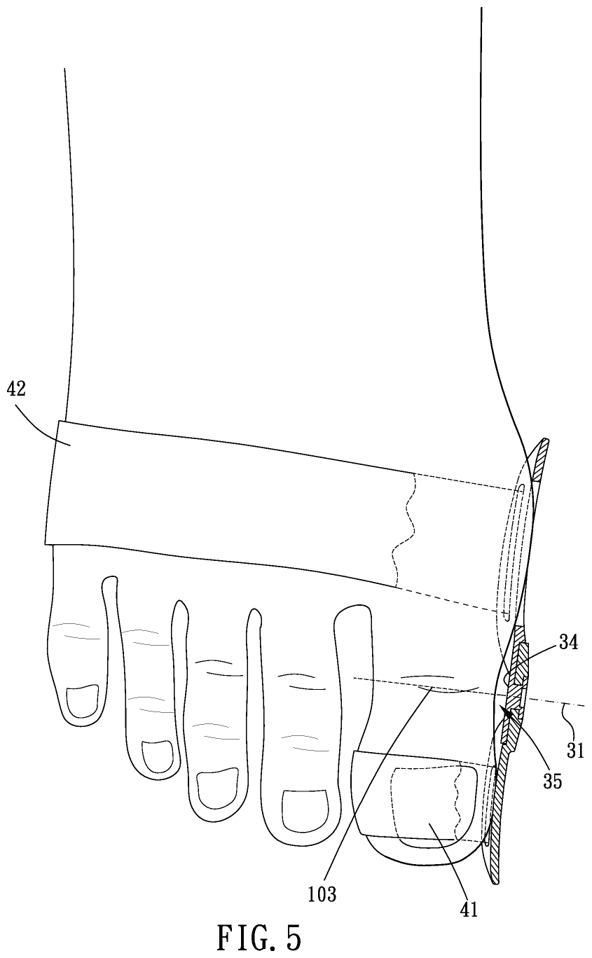

Please refer to FIGS. 1 to 5 for a preferable embodiment of the present invention. An orthosis 1 of the present invention includes a first supporting member 10, a second supporting member 20, a pivot mechanism 30 and an attachment mechanism 40.

The first supporting member 10 is configured to be attached to an inner side of a foot and correspond to a big toe. The second supporting member 20 is configured to be attached to the inner side of the foot and correspond to a metatarsophalangeal (MTP) joint 101 of the big toe. The first supporting member 10 and the second supporting member 20 are rotatably connected with the pivot mechanism 30. The pivot mechanism 30 has a pivot axis 31 configured to correspond to a distal interphalangeal (DIP) joint 102 of the big toe. The attachment mechanism 40 is configured to attach the first supporting member 10 and the second supporting member 20 to the foot. Therefore, the MTP joint 101 and the DIP joint 102 of the big toe are rotatable during wearing the orthosis 1 so that the orthosis 1 is comfortable for wearing.

A length D1 from an endmost point of the second supporting member 20 to the pivot axis 31 is 1.1 to 1.8 times a length D2 from an endmost point of the first supporting member 10 to the pivot axis 31 so that the first supporting member 10 and the second supporting member 20 can correspond to appropriate positions of the inner side of the foot. In this embodiment, the first supporting member 10 and the second supporting member 20 are arcuate boards which can fit a shape of the inner side of the foot. The first supporting member 10 and the second supporting member 20 may be made from plastic, fabric, rubber or the like. The second supporting member 20 includes a receiving portion 21 which is configured to receive the MTP joint 101 of the big toe therewithin. In this embodiment, the receiving portion 21 is a perforation which allows the MTP joint 101 of the big toe to be rotatable without pressure and friction from the second supporting member 20. In other embodiments, the receiving portion may be a recession or an arcuate concave not perforative.

The DIP joint 102 defines a joint axis 103, and a distance between a segment of the pivot axis 31 passing through the pivot mechanism 30 and the joint axis 103 is smaller than or equal to 5 mm when the orthosis 1 is attached to the inner side of the foot. Therefore, the pivot axis 31 preferably corresponds to the joint axis 103 and the orthosis 1 is able to be pivoted synchronously with the big toe.

Specifically, the attachment mechanism 40 includes a first attachment portion 41 and a second attachment portion 42 which are connected with the first supporting member 10 and the second supporting member 20, respectively, and the first and second attachment portions 41, 42 are respectively spaced at least 5 mm apart from the pivot axis 31 in a radial direction of the pivot axis 31 so that the DIP joint 102 of the big toe can rotate without interference of the first and second attachment portions 41, 42 and the orthosis 1 is comfortable for wearing. The first attachment portion 41 and the second attachment portion 42 may be elastic bandages, Velcro strips or strips with buckles or the like for easy adjustment.

At least one of the first and second supporting members 10, 20 includes at least one assembling groove 11, 22, and at least one of the first and second attachment portions 41, 42 penetrates through the at least one assembling groove 11, 22 for adjustable and detachable attachment of the orthosis 1 to the foot. At least one of the first and second supporting members 10, 20 further includes at least one concave portion 12 disposed around the at least one assembling groove 11, 22 and extending to an edge of one of the first and second supporting members 10, 20, and a depth of the at least one concave portion 12 is larger than or equal to a thickness of one of the first and second attachment portions 41, 42. In this embodiment, the first and second supporting members 10, 20 respectively includes two of said assembling grooves 11, 22, and the first supporting member 10 includes two of said concave portions 12 respectively around the two of said assembling grooves 11 so that the first attachment portion 41 does not protrude beyond a surface of the first supporting member 10 away from the big toe to provide good wearing comfortability and appearance. However, it may be designed to have any other numbers of the at least one assembling groove and the at least one concave portion according to various requirements.

The pivot mechanism 30 includes a first engaging portion 32 and a second engaging portion 33 which are detachably and rotatably connected with each other. The first engaging portion 32 includes a through hole 321 and an annular step 322 disposed on an inner peripheral wall of the through hole 321, the second engaging portion 33 includes a projection 331 inserted within the through hole 321, the projection 331 has a flange 332 which extends radially therefrom and is flush with or protrudes beyond a circumferential wall of the through hole 321, and the flange 332 is abuttable against the annular step 322. The pivot mechanism 30 has a simple structure and is easy to be processed and assembled. Preferably, the first engaging portion 32 further includes a notch 323 radially communicated with the through hole 321, the flange 332 includes a mouth 333 disposed thereon, the flange 332 and the annular step 322 urge with each other to assemble the first engaging portion 32 with the second engaging portion 33, and the notch 323 and the mouth 333 are located at opposite sides of the pivot mechanism 30 when the first supporting member 10 is in line with the second supporting member 20, which provides stable engagement between the first and second engaging portions 32, 33. Moreover, with the notch 323 and the mouth 333, the annular step 322 and the flange 332 are easy to elastically deform during urging with each other for easy assembling. The pivot mechanism 30 further includes a plane 34 configured to face toward the DIP joint 102 of the big toe, and the plane 34 and the DIP joint 102 of the big toe define a gap 35 therebetween, which allows the DIP joint 102 to rotate smoothly. In this embodiment, the first engaging portion 32 and the second engaging portion 33 are integrally form as parts of the first supporting member 10 and the second supporting member 20, respectively, for preferable structural strength and easy processing. However, the first and second engaging portions may be respectively and additionally attached to the first and second supporting members.

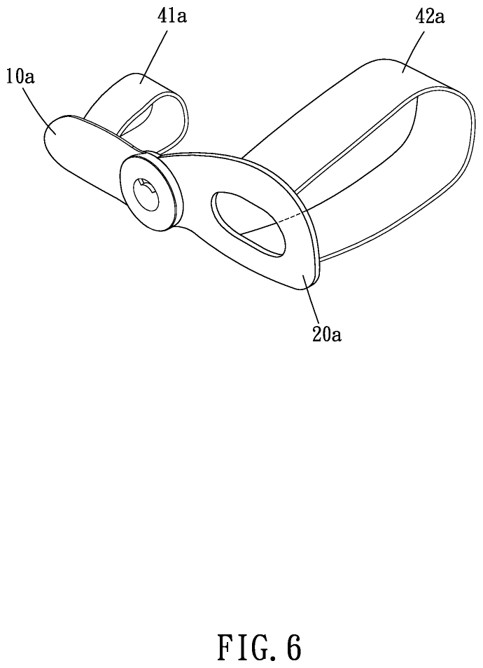

Please refer to FIG. 6 which illustrates another preferable embodiment of the present invention. The first attachment portion 41a and the second attachment portion 42a are fixedly connected with the first supporting member 10a and the second supporting member 20a, respectively. Preferably, the first attachment portion 41a, the second attachment portion 42a, the first supporting member 10a and the second supporting member 20a are made of elastic materials (such as rubber, fabrics, plastics and silicone rubber) for well-fit of a shape of the foot and comfortable wearing.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. An orthosis, including:
a first supporting member, configured to be attached to an inner side of a foot and correspond to a big toe;
a second supporting member, configured to be attached to the inner side of the foot and correspond to a metatarsophalangeal (MTP) joint of the big toe;
a pivot mechanism with which the first supporting member and the second supporting member are rotatably connected, the pivot mechanism having a pivot axis configured to correspond to a distal interphalangeal (DIP) joint of the big toe;
an attachment mechanism, configured to attach the first supporting member and the second supporting member to the foot.

2. The orthosis of claim 1, wherein a length from an endmost point of the second supporting member to the pivot axis is 1.1 to 1.8 times a length from an endmost point of the first supporting member to the pivot axis.

3. The orthosis of claim 1, wherein the DIP joint defines a joint axis, and a distance between a segment of the pivot axis passing through the pivot mechanism and the joint axis is smaller than or equal to 5 mm when the orthosis is attached to the inner side of the foot.

4. The orthosis of claim 1, wherein the attachment mechanism includes a first attachment portion and a second attachment portion which are connected with the first supporting member and the second supporting member, respectively, and the first and second attachment portions are respectively spaced at least 5 mm apart from the pivot axis in a radial direction of the pivot axis.

5. The orthosis of claim 4, wherein the first attachment portion and the second attachment portion are fixedly connected with the first supporting member and the second supporting member, respectively.

6. The orthosis of claim 1, wherein the second supporting member includes a receiving portion which is configured to receive the MTP joint of the big toe therewithin.

7. The orthosis of claim 1, wherein the pivot mechanism includes a first engaging portion and a second engaging portion which are detachably and rotatably connected with each other.

8. The orthosis of claim 7, wherein the first engaging portion includes a through hole and an annular step disposed on an inner peripheral wall of the through hole, the second engaging portion includes a projection inserted within the through hole, the projection has a flange which extends radially therefrom and is flush with or protrudes beyond a circumferential wall of the through hole, and the flange is abuttable against the annular step.

9. The orthosis of claim 8, wherein the first engaging portion further includes a notch radially communicated with the through hole, the flange includes a mouth disposed thereon, the flange and the annular step urge with each other to assemble the first engaging portion with the second engaging portion, and the notch and the mouth are located at opposite sides of the pivot mechanism when the first supporting member is in line with the second supporting member.

10. The orthosis of claim 9, wherein a length from an endmost point of the second supporting member to the pivot axis is 1.1 to 1.8 times a length from an endmost point of the first supporting member to the pivot axis; the DIP joint defines a joint axis, and a distance between a segment of the pivot axis passing through the pivot mechanism and the joint axis is smaller than or equal to 5 mm when the orthosis is attached to the inner side of the foot; the attachment mechanism includes a first attachment portion and a second attachment portion which are connected with the first supporting member and the second supporting member, respectively, and the first and second attachment portions are respectively spaced at least 5 mm apart from the pivot axis in a radial direction of the pivot axis; the second supporting member includes a receiving portion which is configured to receive the MTP joint of the big toe therewithin; at least one of the first and second supporting members includes at least one assembling groove, and at least one of the first and second attachment portions penetrates through the at least one assembling groove; at least one of the first and second supporting members further includes at least one concave portion disposed around the at least one assembling groove and extending to an edge of one of the first and second supporting members, and a depth of the at least one concave portion is larger than or equal to a thickness of one of the first and second attachment portions; the pivot mechanism further includes a plane configured to face toward the DIP joint of the big toe, and the plane and the DIP joint of the big toe define a gap therebetween.
